(19) United States
(12) Patent Application Publication
(10) Pub. No.: US 2007/0099912 A1
(43) Pub. Date: May 3, 2007

(54) Pyrrolo[2,3-F] and [3,2-F]Isoquinolinone derivatives as 5-hydroxytryptamine-6 ligands

(75) Inventors: Ping Zhou, Plainsboro, NJ (US); Yanfang Li, Lawrenceville, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/588,610

(22) Filed: Oct. 27, 2006

Related U.S. Application Data
(60) Provisional application No. 60/731,215, filed on Oct. 28, 2005.

(51) Int. Cl. A61K 31/541; A61K 31/5377; A61K 31/496; A61K 31/4745; C07D 471/02

(52) U.S. Cl. 514/227.8; 514/232.8; 514/253.03; 514/291; 544/60; 544/126; 544/381; 546/82

(57) ABSTRACT

The present invention provides a compound of formula I or II and the use thereof in the therapeutic treatment of a central nervous system disorder related to or affected by the 5-HT6 receptor.

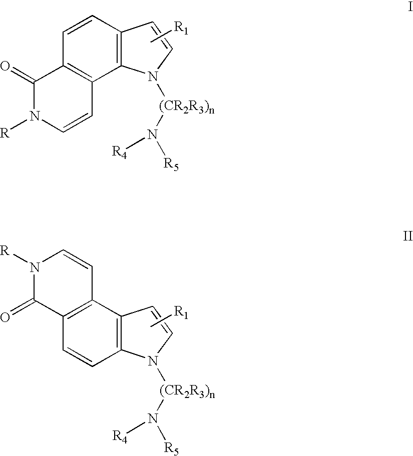

### BACKGROUND OF THE INVENTION

This application claims the benefit under 35 U.S.C. §119(e) to co-pending U.S. provisional application No. 60/731,215, filed Oct. 28, 2005, which is hereby incorporated by reference in its entirety.

Serotonin (5-Hydroxytryptamine)(5-HT) receptors play a critical role in many physiological and behavioral functions in humans and animals. These functions are mediated through various 5-HT receptors distributed throughout the body. There are now approximately fifteen different human 5-HT receptor subtypes that have been cloned, many with well-defined roles in humans. The 5-HT6 receptor was first cloned from rat tissue in 1993 (Monsma, F. J.; Shen, Y.; Ward, R. P.; Hamblin, M. W. Molecular Pharmacology 1993, 43, 320-327) and subsequently from human tissue (Kohen, R.; Metcalf, M. A.; Khan, N.; Druck, T.; Huebner, K.; Sibley, D. R. Journal of Neurochemistry 1996, 66, 47-56). The receptor is a G-protein coupled receptor (GPCR) positively coupled to adenylate cyclase (Ruat, M.; Traiffort, E.; Arrang, J-M.; Tardivel-Lacombe, L.; Diaz, L.; Leurs, R.; Schwartz, J-C. Biochemical Biophysical Research Communications 1993, 193, 268-276). The receptor is found almost exclusively in the central nervous system (CNS) areas both in rat and in human. In situ hybridization studies of the 5-HT6 receptor in rat brain using mRNA indicate principal localization in the areas of 5-HT projection including striatum, nucleus accumbens, olfactory tubercle, and hippocampal formation (Ward, R. P.; Hamblin, M. W.; Lachowicz, J. E.; Hoffman, B. J.; Sibley, D. R.; Dorsa, D. M. Neuroscience 1995, 64, 1105-1111).

There are many potential therapeutic uses for 5-HT6 ligands in humans based on direct effects and on indications from available scientific studies. These studies include the localization of the receptor, the affinity of ligands with known in vivo activity, and various animal studies conducted so far.

One potential therapeutic use of modulators of 5-HT6 receptor function is in the enhancement of cognition and memory in human diseases such as Alzheimer's Disease. The high levels of receptor found in important structures in the forebrain, including the caudate/putamen, hippocampus, nucleus accumbens, and cortex suggest a role for the receptor in memory and cognition since these areas are known to play a vital role in memory (Gerard, C.; Martres, M.-P.; Lefevre, K.; Miquel, M. C.; Verge, D.; Lanfumey, R.; Doucet, E.; Hamon, M.; El Mestikawy, S. Brain Research, 1997, 746, 207-219). The ability of known 5-HT6 receptor ligands to enhance cholinergic transmission also supported the potential cognition use (Bentley, J. C.; Boursson, A.; Boess, F. G.; Kone, F. C.; Marsden, C. A.; Petit, N.; Sleight, A. J. British Journal of Pharmacology, 1999, 126(7), 1537-1542). Studies have found that a known 5-HT6 selective antagonist significantly increased glutamate and aspartate levels in the frontal cortex without elevating levels of noradrenaline, dopamine, or 5-HT. This selective elevation of neurochemicals known to be involved in memory and cognition strongly suggests a role for 5-HT6 ligands in cognition (Dawson, L. A.; Nguyen, H. Q.; Li, P. British Journal of Pharmacology, 2000, 130(1), 23-26). Animal studies of memory and learning with a known selective 5-HT6 antagonist have found positive indications (Rogers, D. C.; Hatcher, P. D.; Hagan, J. J. Society of Neuroscience, Abstracts 2000, 26, 680 and Foley, A. G. et al, Neuropsychopharmacology, 2004, 29(1), 93-100).

A related potential therapeutic use for 5-HT6 ligands is the treatment of attention deficit disorders (ADD, also known as Attention Deficit Hyperactivity Disorder or ADHD) in both children and adults. Because 5-HT6 antagonists appear to enhance the activity of the nigrostriatal dopamine pathway and because ADHD has been linked to abnormalities in the caudate (Ernst, M; Zametkin, A. J.; Matochik, J. H.; Jons, P. A.; Cohen, R. M. Journal of Neuroscience 1998, 18(15), 5901-5907), 5-HT6 antagonists may attenuate attention deficit disorders.

Early studies examining the affinity of various CNS ligands with known therapeutic utility or a strong structural resemblance to known drugs suggests a role for 5-HT6 ligands in the treatment of schizophrenia and depression. For example, clozapine (an effective clinical antipsychotic) has high affinity for the 5-HT6 receptor subtype. Also, several clinical antidepressants have high affinity for the receptor as well and act as antagonists at this site (Branchek, T. A.; Blackburn, T. P. Annual Reviews in Pharmacology and Toxicology 2000, 40, 319-334).

Further, recent in vivo studies in rats indicate 5-HT6 modulators may be useful in the treatment of movement disorders including epilepsy (Stean, T.; Routledge, C.; Upton, N. British Journal of Pharmacology 1999, 127 Proc. Supplement 131 P and Routledge, C.; Bromidge, S. M.; Moss, S. F.; Price, G. W.; Hirst, W.; Newman, H.; Riley, G.; Gager, T.; Stean, T.; Upton, N.; Clarke, S. E.; Brown, A. M. British Journal of Pharmacology 2000, 130(7), 1606-1612).

Taken together, the above studies strongly suggest that compounds which are 5-HT6 receptor modulators, i.e. ligands, may be useful for therapeutic indications including: the treatment of diseases associated with a deficit in memory, cognition, and learning such as Alzheimer's and attention deficit disorder; the treatment of personality disorders such as schizophrenia; the treatment of behavioral disorders, e.g., anxiety, depression and obsessive compulsive disorders; the treatment of motion or motor disorders such as Parkinson's disease and epilepsy; the treatment of diseases associated with neurodegeneration such as stroke and head trauma; or withdrawal from drug addiction including addiction to nicotine, alcohol, and other substances of abuse.

Therefore, it is an object of this invention to provide compounds which are useful as therapeutic agents in the treatment of a variety of central nervous system disorders related to or affected by the 5-HT6 receptor.

It is another object of this invention to provide therapeutic methods and pharmaceutical compositions useful for the treatment of central nervous system disorders related to or affected by the 5-HT6 receptor.

It is a feature of this invention that the compounds provided may also be used to further study and elucidate the 5-HT6 receptor.

### SUMMARY OF THE INVENTION

The present invention provides a pyrroloisoquinolinone compound of formula I or II 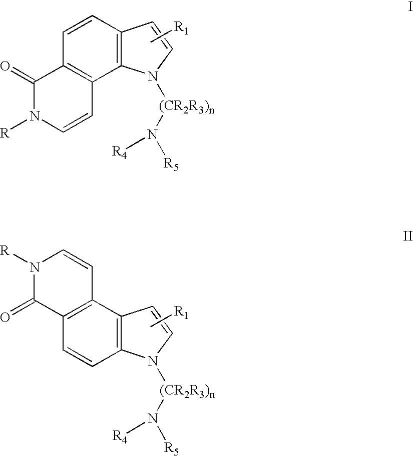
wherein
R is H or a C₁-C₆alkyl, C₁-C₄arylalkyl, C₁-C₄heteroarylalkyl, C₃-C₇cycloalkylalkyl, cycloheteroalkyl, aryl or heteroaryl group each optionally substituted;
R₁ is H, halogen or a C₁-C₆alkoxy or C₁-C₄alkyl group each optionally substituted;
R₂ and R₃ are each independently H or a C₁-C₆alkyl or C₃-C₇cycloalkyl group each optionally substituted or R₂ may be taken together with R₄ to form an optionally substituted 5- to 8-membered ring;
n is 0 or an integer of 1, 2 or 3; and
R₄ and R₅ are each independently H or a C₁-C₆alkyl or C₃-C₇cycloalkyl group each optionally substituted or R₄ and R₅ may be taken together with the atom to which they are attached to form an optionally substituted 5- to 8-membered ring optionally containing an additional heteroatom selected from O, S, or NR; or
   a stereoisomer thereof or a pharmaceutically acceptable salt thereof.

The present invention also provides methods and compositions useful in the treatment of central nervous system disorders.

### DETAILED DESCRIPTION OF THE INVENTION

The 5-hydroxytryptamine-6 (5-HT6) receptor has been identified by molecular cloning. Its ability to bind a wide range of therapeutic compounds used in psychiatry, coupled with its intriguing distribution in the brain has stimulated significant interest in new compounds which are capable of interacting with or affecting said receptor. Significant efforts are being made to understand the possible role of the 5-HT6 receptor in psychiatry, cognitive dysfunction, motor function and control, memory, mood and the like. To that end, compounds which demonstrate a binding affinity for the 5-HT6 receptor are earnestly sought both as an aid in the study of the 5-HT6 receptor and as potential therapeutic agents in the treatment of central nervous system disorders, for example see C. Reavill and D. C. Rogers, Current Opinion in Investigational Drugs, 2001, 2(1):104-109, Pharma Press Ltd.

Surprisingly, it has now been found that a pyrroloisoquinolinone compound of of formula I or II demonstrates 5-HT6 affinity along with significant sub-type selectivity. Advantageously, said formula I or II compounds are effective therapeutic agents for the treatment of central nervous system (CNS) disorders associated with or affected by the 5-HT6 receptor. Accordingly, the present invention provides pyrroloisoquinolinone compounds of formula I or II 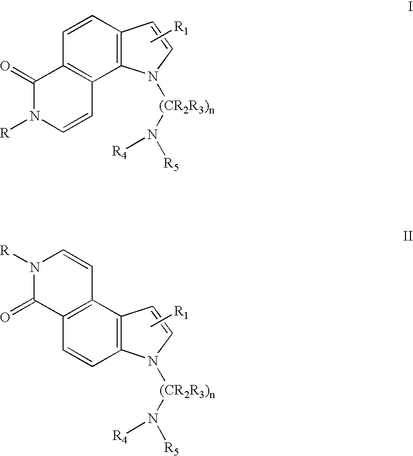
wherein
R is H or a C₁-C₆alkyl, C₁-C₄arylalkyl, C₁-C₄heteroarylalkyl, C₃-C₇cycloalkylalkyl, cycloheteroalkyl, aryl or heteroaryl group each optionally substituted;
R₁ is H, halogen or a C₁-C₆alkoxy or C₁-C₄alkyl group each optionally substituted;
R₂ and R₃ are each independently H or a C₁-C₆alkyl or C₃-C₇cycloalkyl group each optionally substituted or R₂ may be taken together with R₄ to form an optionally substituted 5- to 8-membered ring;
n is 0 or an integer of 1, 2 or 3; and
R₄ and R₅ are each independently H or a C₁-C₆alkyl or C₃-C₇cycloalkyl group each optionally substituted or R₄ and R₅ may be taken together with the atom to which they are attached to form an optionally substituted 5 to 8-membered ring optionally containing an additional heteroatom selected from O, S, or NR; or
   a stereoisomer thereof or a pharmaceutically acceptable salt thereof.

As used in the specification and claims, the term halogen designates F, Cl, Br or I and the term cycloheteroalkyl designates a five- to seven-membered cycloalkyl ring system containing 1 or 2 heteroatoms, which may be the same or different, selected from N, O or S and optionally containing one double bond. Exemplary of the cycloheteroalkyl ring systems included in the term as designated herein are the following rings wherein X is NR′, O or S; and R′ is H or an optional substituent as described hereinbelow:

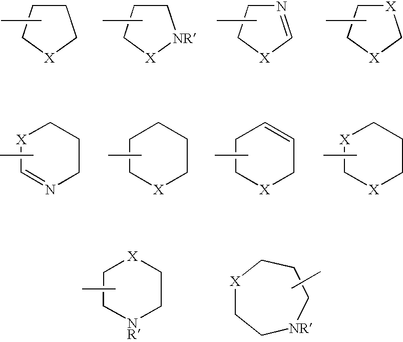

Similarly, as used in the specification and claims, the term heteroaryl designates a five- to ten-membered aromatic ring system containing 1, 2 or 3 heteroatoms, which may be the same or different, selected from N, O or S. Such heteroaryl ring systems include pyrrolyl, azolyl, oxazolyl, thiazolyl, imidazolyl, furyl, thienyl, quinolinyl, isoquinolinyl, indolyl, benzothienyl, benzofuranyl, benzisoxazolyl or the like. The term aryl designates a carbocyclic aromatic ring system such as phenyl, naphthyl, anthracenyl or the like.

In the specification and claims, when the terms C₁-C₆alkyl, C₂-C₆alkenyl, C₂-C₆alkynyl, C₃-C₇cycloalkyl, cycloheteroalkyl, aryl or heteroaryl are designated as being optionally substituted, the substituent groups which are optionally present may be one or more of those customarily employed in the development of pharmaceutical compounds, or the modification of such compounds, to influence their structure/activity, persistence, absorption, stability or other beneficial property. Specific examples of such substituents include halogen atoms, nitro, cyano, thiocyanato, cyanato, hydroxyl, alkyl, haloalkyl, alkoxy, haloalkoxy, amino, alkylamino, dialkylamino, formyl, alkoxycarbonyl, carboxyl, alkanoyl, alkylthio, alkylsuphinyl, alkylsulphonyl, carbamoyl, alkylaminocarbonyl, phenyl, phenoxy, benzyl, benzyloxy, heteroaryl, indolyl, heterocyclyl or cycloalkyl groups, preferably halogen atoms or lower alkyl or lower alkoxy groups. Typically, 0-3 substituents may be present. When any of the foregoing substituents represents or contains an alkyl substituent group, this may be linear or branched and may contain up to 12, preferably up to 6, more preferably up to 4 carbon atoms.

Pharmaceutically acceptable salts may be any acid addition salt formed by a compound of formula I and a pharmaceutically acceptable acid such as phosphoric, sulfuric, hydrochloric, hydrobromic, citric, maleic, malonic, mandelic, succinic, fumaric, acetic, lactic, nitric, sulfonic, p-toluene sulfonic, methane sulfonic acid or the like.

Compounds of the invention include esters, carbamates or other conventional prodrug forms, which in general, are functional derivatives of the compounds of the invention and which are readily converted to the inventive active moiety in vivo. Correspondingly, the method of the invention embraces the treatment of the various conditions described hereinabove with a compound of formula I or with a compound which is not specifically disclosed but which, upon administration, converts to a compound of formula I in vivo. Also included are metabolites of the compounds of the present invention defined as active species produced upon introduction of these compounds into a biological system.

Compounds of the invention may exist as one or more stereoisomers. The various stereoisomers include enantiomers, diastereomers, atropisomers and geometric isomers. One skilled in the art will appreciate that one stereoisomer may be more active or may exhibit beneficial effects when enriched relative to the other stereoisomer(s) or when separated from the other stereoisomer(s). Additionally, the skilled artisan knows how to separate, enrich or selectively prepare said stereoisomers. Accordingly, the present invention comprises compounds of formula I or II, the stereoisomers thereof and the pharmaceutically acceptable salts thereof. The compounds of the invention may be present as a mixture of stereoisomers, individual stereoisomers, or as an optically active or enantiomerically pure form.

Preferred compounds of the invention are those compounds of formula I or II wherein R₁ is H. Another group of preferred compounds of the invention are those compounds of formula I or II wherein n is 2 and R₂ and R₃ are H. Also preferred are those compounds of formula I or II wherein R is H, arylalkyl or heteroarylalkyl.

More preferred compounds of the invention are those compounds of formula I or II wherein R₄ and R₅ are each independently H or C₁-C₄alkyl. Another group of more preferred compounds of formula I or II are those compounds wherein R₁, R₂ and R₃ are H and n is 2.

Among the preferred compounds of the invention are: 7-benzyl-1-[2-(dimethylamino)ethyl]-1,7-dihydro-6H-pyrrolo[2,3-f]isoquinolin-6-one; 1-(2-aminoethyl)-7-benzyl-1,7-dihydro-6H-pyrrolo[2,3-f]isoquinolin-6-one; 1-(2-aminoethyl)-7-(thien-3-ylmethyl)-1 ,7-dihydro-6H-pyrrolo[2,3-f]isoquinolin-6-one; 1-(2-aminoethyl)-7-(cyclohexylmethyl)-1,7-dihydro-6H-pyrrolo[2,3-f]isoquinolin-6-one; 3-(2-aminoethyl)-7-(4-fluorobenzyl)-3,7-dihydro-6H-pyrrolo[3,2-f]isoquinolin-6-one; 3-[2-(dimethylamino)ethyl]-7-(4-fluorobenzyl)-3,7-dihydro-6H-pyrrolo[3,2-f]isoquinolin-6-one; 7-(4-fluorobenzyl)-3-(2-pyrrolidin-1-ylethyl)-3,7-dihydro-6H-pyrrolo[3,2-f]isoquinolin-6-one; 3-[3-(dimethylamino)propyl]-7-(4-fluorobenzyl)-3,7-dihydro-6H-pyrrolo[3,2-f]isoquinolin-6-one; or 1-(2-aminoethyl)-7-(1-naphthylmethyl)-1,7-dihydro-6H-pyrrolo[2,3-f]isoquinolin-6-one; 1-(2-aminoethyl)-7-(quinolin-8-ylmethyl)-1,7-dihydro-6H-pyrrolo[2,3-f]isoquinolin-6-one; 1-(2-aminoethyl)-7-[(6-chloroimidazo[2,1-b][1,3]thiazol-5-yl)methyl]-1,7-dihydro-6H-pyrrolo[2,3-f]isoquinolin-6-one; 1-(2-aminoethyl)-7-[(3-chloro-1-benzothien-2-yl)methyl]-1,7-dihydro-6H-pyrrolo[2,3-f]isoquinolin-6-one; 1-(2-aminoethyl)-7-(2-thienylmethyl)-1 ,7-dihydro-6H-pyrrolo[2,3-f]isoquinolin-6-one; 1-(2-aminoethyl)-7-(2-thienylmethyl)-1 ,7-dihydro-6H-pyrrolo[2,3-f]isoquinolin-6-one; 1-(2-aminoethyl)-7-(2-chlorobenzyl)-1,7-dihydro-6H-pyrrolo[2,3-f]isoquinolin-6-one; 1-(2-aminoethyl)-7-(3-chlorobenzyl)-1 ,7-dihydro-6H-pyrrolo[2,3-f]isoquinolin-6-one;

a stereoisomer thereof; or

a pharmaceutically acceptable salt thereof.

Compounds of formula I or II may be prepared using conventional synthetic methods and, if required, standard separation or isolation techniques. For example, compounds of formula I wherein R is benzyl (Ia) may be prepared by coupling a 7-bromoindole of formula III with methyl acrylate to give methyl indolylpropenoate (Cerri, A. et al, J. Heterocyclic Chem. 1993, 30, 1581) and hydrolyzing said propenoate in the presence of a base to give the corresponding carboxylic acid of formula IV, reacting the formula IV acid with NaN₃ to obtain the activated indolylpropenoyl azide of formula V, heating said formula V azide to effect ring formation to give a pyrroloisoquinolinone of formula VI, selectively benzylating said formula VI isoquinolinone to give the intermediate compound of formula VII and alkylating said formula VII intermediate with an aminoalkyl chloride of formula VIII to obtain the desired compound of formula Ia. The reaction sequence is shown in flow diagram I wherein Me is methyl and Bu is butyl.

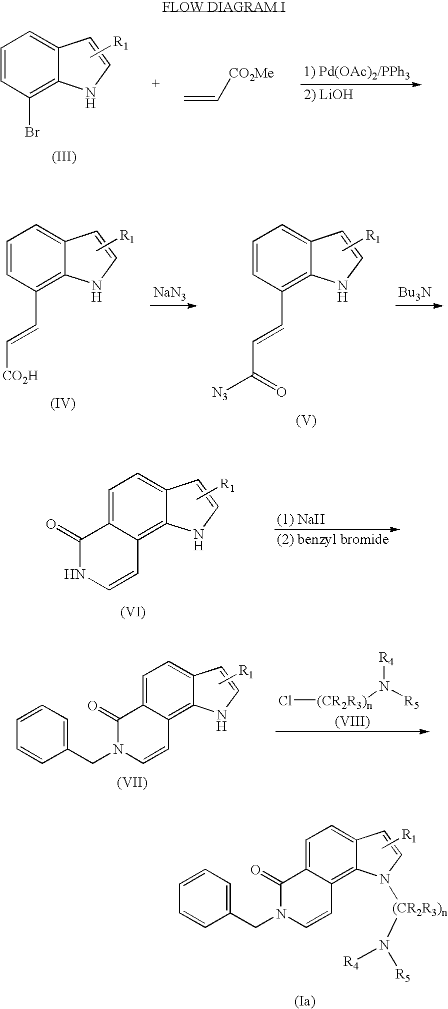

Correspondingly, compounds of formula II may be prepared, in a manner similar to that described hereinabove in flow diagram I, by employing a 4-bromoindole derivative as starting material in place of the 7-bromoindole of formula III. The reactions are illustrated in flow diagram II.

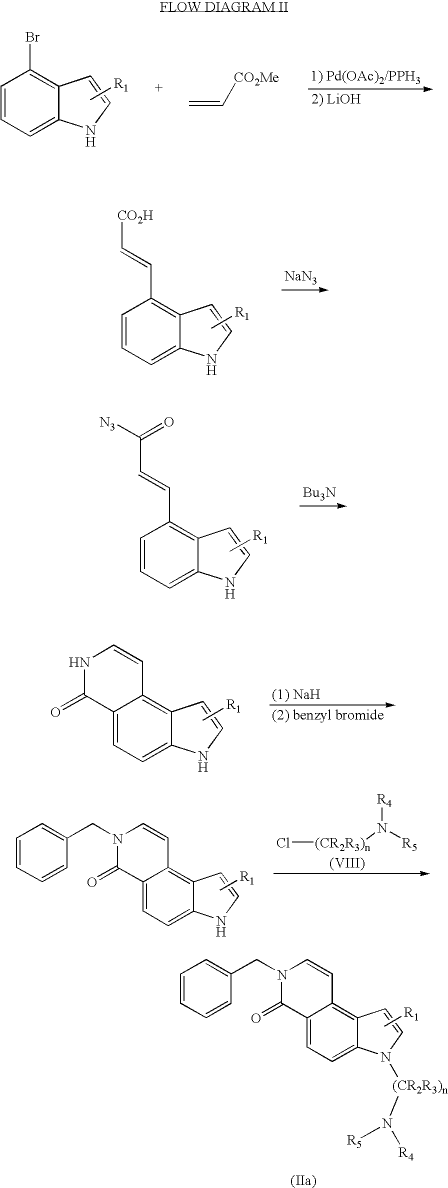

Advantageously, the formula I and formula II compounds of the invention are useful for the treatment of CNS disorders relating to or affected by the 5-HT6 receptor including mood, personality, behavioral, psychiatric, cognitive, neurodegenerative, or the like disorders, for example Alzheimer's disease, Parkinson's disease, attention deficit disorder, anxiety, epilepsy, depression, obsessive compulsive disorder, sleep disorders, neurodegenerative disorders (such as head trauma or stroke), feeding disorders (such as anorexia or bulimia), schizophrenia, memory loss, disorders associated with withdrawal from drug or nicotine abuse, or the like or certain gastrointestinal disorders such as irritable bowel syndrome. Accordingly, the present invention provides a method for the treatment of a disorder of the central nervous system related to or affected by the 5-HT6 receptor in a patient in need thereof which comprises providing said patient a therapeutically effective amount of a compound of formula I or formula II as described hereinabove. The compounds may be provided by oral or parenteral administration or in any common manner known to be an effective administration of a therapeutic agent to a patient in need thereof.

The term “providing” as used herein with respect to providing a compound or substance embraced by the invention, designates either directly administering such a compound or substance, or administering a prodrug, derivative or analog which forms an equivalent amount of the compound or substance within the body.

The therapeutically effective amount provided in the treatment of a specific CNS disorder may vary according to the specific condition(s) being treated, the size, age and response pattern of the patient, the severity of the disorder, the judgment of the attending physician or the like. In general, effective amounts for daily oral administration may be about 0.01 to 1,000 mg/kg, preferably about 0.5 to 500 mg/kg and effective amounts for parenteral administration may be about 0.1 to 100 mg/kg, preferably about 0.5 to 50 mg/kg.

In actual practice, the compounds of the invention are provided by administering the compound or a precursor thereof in a solid or liquid form, either neat or in combination with one or more conventional pharmaceutical carriers or excipients. Accordingly, the present invention provides a pharmaceutical composition which comprises a pharmaceutically acceptable carrier and an effective amount of a compound of formula I or formula II as described hereinabove.

Solid carriers suitable for use in the composition of the invention include one or more substances which may also act as flavoring agents, lubricants, solubilizers, suspending agents, fillers, glidants, compression aides, binders, tablet-disintegrating agents or encapsulating materials. In powders, the carrier may be a finely divided solid which is in admixture with a finely divided compound of formula 1. In tablets, the formula I compound may be mixed with a carrier having the necessary compression properties in suitable proportions and compacted in the shape and size desired. Said powders and tablets may contain up to 99% by weight of the formula I compound. Solid carriers suitable for use in the composition of the invention include calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Any pharmaceutically acceptable liquid carrier suitable for preparing solutions, suspensions, emulsions, syrups and elixirs may be employed in the composition of the invention. Compounds of formula I or formula II may be dissolved or suspended in a pharmaceutically acceptable liquid carrier such as water, an organic solvent, or a pharmaceutically acceptable oil or fat, or a mixture thereof. Said liquid composition may contain other suitable pharmaceutical additives such as solubilizers, emulsifiers, buffers, preservatives, sweeteners, flavoring agents, suspending agents, thickening agents, coloring agents, viscosity regulators, stabilizers, osmo-regulators, or the like. Examples of liquid carriers suitable for oral and parenteral administration include water (particularly containing additives as above, e.g., cellulose derivatives, preferably sodium carboxymethyl cellulose solution), alcohols (including monohydric alcohols and polyhydric alcohols, e.g., glycols) or their derivatives, or oils (e.g., fractionated coconut oil and arachis oil). For parenteral administration the carrier may also be an oily ester such as ethyl oleate or isopropyl myristate.

Compositions of the invention which are sterile solutions or suspensions are suitable for intramuscular, intraperitoneal or subcutaneous injection. Sterile solutions may also be administered intravenously. Inventive compositions suitable for oral administration may be in either liquid or solid composition form.

For a more clear understanding, and in order to illustrate the invention more clearly, specific examples thereof are set forth hereinbelow. The following examples are merely illustrative and are not to be understood as limiting the scope and underlying principles of the invention in any way.

Unless otherwise stated, all parts are parts by weight. The term NMR designates nuclear magnetic resonance. The terms THF, DMF and EtOAc designate tetrahydrofuran, dimethyl formamide and ethyl acetate, respectively.

### EXAMPLE 1

### Preparation of Methyl 3-(1H-indol-7-yl)prop-2-enoate

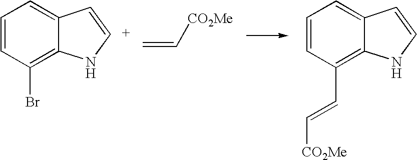

To a solution of 7-bromoindole (7.71 g, 39.3 mmol) in DMF (40 ml) is added methyl acrylate (6.76 g, 78.7 mmol), palladium (II) acetate (0.179 g, 0.786 mmol), triphenyl phospine (0.412 g, 1.57 mmol) and N,N-diisopropylethylamine (6.34 g, 49.2 mmol). After stirring at 100° C. for 3 days, the reaction mixture is cooled to room temperature, treated with aqueous 1N HCl (150 ml) and extracted with ethyl acetate (3×100 ml). The combined organic extracts are washed with aqueous 1N HCl (3×100 ml), brine (300 ml), dried (MgSO₄) and concentrated. The crude product is re-crystallized from MeOH/H₂O to afford the title compound as a yellow solid (6.8 g, 86%); mp 85-86° C.; MS (−) ESI: 200 (M−H)⁻; the compound is identified by ¹H NMR. Elemental Analysis for: C₁₂H₁₁NO₂ 0.3 H₂O Calculated: C, 69.75; H, 5.66; N, 6.78 Found: C, 69.74; H, 5.61; N, 6.54

### EXAMPLE 2

### Preparation of 3-(1H-Indol-7-yl)prop-2-enoic acid

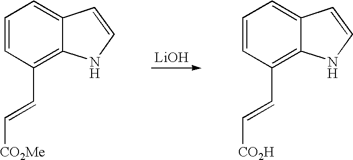

To a solution of methyl 3-(1H-indol-7-yl)prop-2-enoate (6.30 g, 31.5 mmol) in THF (40 ml) is added aqueous 1N LiOH (48 ml). The reaction mixture is stirred at room temperature for 18 hours, cooled in ice-bath and treated with concentrated HCl to pH=2. The solvent is removed on a rotary evaporator. The resulting suspension is extracted with several portions of EtOAc. The combined extracts are washed with brine, dried (MgSO₄) and concentrated. The crude product is re-crystallized from MeOH/H₂O (75/25) to afford the title compound as a light brown solid (5.15 g, 87%); mp 179-181° C.; MS (−) ESI: 186 (M−H)⁻; the compound is identified by ¹H NMR. Elemental Analysis for: C₁₁H₉NO₂ 0.1 H₂O Calculated: C, 69.91; H, 4.91; N, 7.41 Found: C, 69.65; H, 4.94; N, 7.23

### EXAMPLE 3

### Preparation of 3-(1H-Indol-7-yl)prop-2-enoic azide

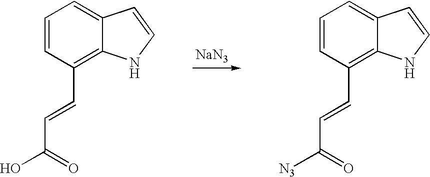

To a solution of triethylamine (4.17 ml, 30.3 mmol) in acetone (24 ml) is added 3-(1H-indol-7-yl)prop-2-enoic acid (5.15 g, 27.5 mmol). The reaction mixture is maintained below 0° C. while a solution of ethyl chloroformate (4.06 g, 37.5 mmol) in acetone (24 ml) is added dropwise. After stirring the mixture at 0° C. for 1 hour, a solution of sodium azide (2.69 g, 41.31 mmol) in H₂O (10 ml) is added in portions. The reaction is stirred at 0° C. for 1 hour. The insoluble material is removed by filtration and the filtrate is concentrated. The residue is dissolved in Et₂O, washed with water, saturated aqueous NaHCO₃, brine, dried over (MgSO₄) and concentrated. The crude product is purified by chromatography (silica gel, EtOAc/hexane: 10/90-30/70) and proceeded to next step.

### EXAMPLE 4

### Preparation of 1,7-Dihydro-6H-pyrrolo[2,3-f]isoquinolin-6-one

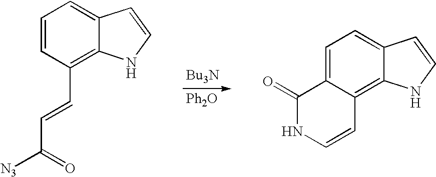

To a solution of Bu₃N (8.88 ml, 37.4 mmol) in Ph₂O (60 ml) at 245° C. is added a solution of 3-(1 H-indol-7-yl)prop-2-enoic azide (5.28 g, 24.9 mmol) in Ph₂O (60 ml). After stirring at 220° C. for 30 minutes, the reaction mixture is cooled to room temperature and treated with hexane. The resulting precipitate is collected by filtration. The crude product is purified by chromatography (silica gel, MeOH/CH₂Cl₂: 5/95) to afford a tan solid (1.1 g, 24%); mp 244-245° C.; MS (+) ESI: 185 (M+H)⁺; the compound is identified by ¹H NMR. Elemental Analysis for: C₁₁H₈N₂O 0.2 H₂O 0.4 EtOAc Calculated: C, 67.85; H, 5.24; N, 12.56 Found: C, 67.97; H, 5.20; N, 12.52

### EXAMPLE 5

### Preparation of 7-Benzyl-1,7-dihydro-6H-pyrrolo[2,3-f]isoquinolin-6-one

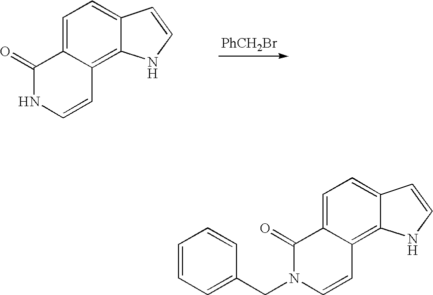

To a solution of 1,7-dihydro-6H-pyrrolo[2,3-f]isoquinolin-6-one (1.10 g, 5.98 mmol) in DMF (10 ml) is added K₂CO₃ (1.65 g, 12.0 mmol) and benzyl bromide (1.02 g, 5.98 mmol). After stirring at room temperature for 18 hours, the reaction mixture is diluted with water and EtOAc and the insoluble material is removed by filtration. The filtrate is extracted with several portions of EtOAc. The combined organic extracts are washed with aqueous LiCl (10%), brine, dried (MgSO₄) and concentrated. The residue is purified by chromatography (silica gel, EtOAc/hexane: 50/50) to afford the title compound as a yellow solid (0.82 g, 50%) mp 211-213° C.; MS (+) ESI: 273 (M−H)⁻; the compound is identified by ¹H NMR. Elemental Analysis for: C₁₈H₁₄N₂O Calculated: C, 78.81; H, 5.14; N, 10.21 Found: C, 78.42; H, 5.27; N, 9.97

### EXAMPLE 6

### Preparation of 7-Benzyl-1-[2-(dimethylamino)ethyl]-1.7-dihydro-6H-pyrrolo[2,3-f]isoquinolin-6-one hydrochloride

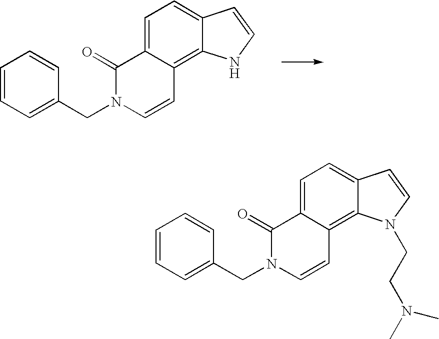

To a solution of 7-benzyl-1,7-dihydro-6H-pyrrolo[2,3-f]isoquinolin-6-one (0.140 g, 0.50 mmol) in CH₃CN (5 ml) is added NaOH (0.072 g, 1.80 mmol). After stirring the reaction mixture at room temperature for 30 min, tetrabutylammonium hydrogensulfate (0.0068 g, 0.02 mmol) and N-(2-chloroethyl)-N,N-dimethylamine hydrochloride (0.079 g, 0.55 mmol) are added. After refluxing for 24 hours, the resultant suspension is cooled to room temperature and the solid is filtered off. The filtrate is concentrated and the crude product is purified by chromatography (silica gel, 2N NH₃ in MeOH/CH₂Cl₂: 5/95) to afford a white solid (0.089 g). The white solid is treated with 2N HCl/Et₂O to afford the title compound as mono salt; mp 100-102° C.; MS (+) ESI: 346 (M+H)⁺; the compound is identified by ¹H NMR. Elemental Analysis for: C₂₂H₂₃N₃O 1.0 HCl 3.0 H₂O Calculated: C, 60.61; H, 6.94; N, 9.64 Found: C, 60.63; H, 6.17; N, 9.64

### EXAMPLE 7

### Preparation of 1-(2-Aminoethyl)-7-benzyl-1,7-dihydro-6H-pyrrolo[2,3-f]isoquinolin-6-one hydrochloride

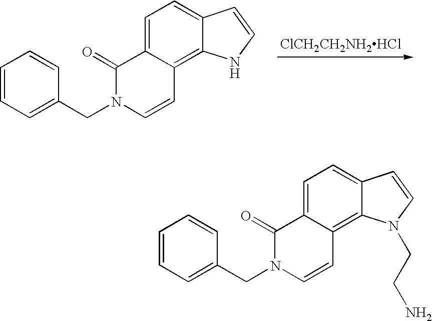

To a solution of 7-benzyl-1,7-dihydro-6H-pyrrolo[2,3-f]isoquinolin-6-one (0.25 g, 0.91 mmol) in CH₃CN (4 ml) is added NaOH (0.130 g, 3.28 mmol). After stirring reaction mixture at room temperature for 30 min, tetrabutylammonium hydrogensulfate (0.012 g, 0.036 mmol) and 2-chloroethylamine hydrochloride (0.160 g, 1.00 mmol) are added. The resultant suspension is refluxed for 3 days. After cooling the reaction mixture to room temperature, the solid is filtered off. The filtrate is concentrated and the crude product is purified by chromatography (silica gel, 2N NH₃ in MeOH/CH₂Cl₂: 5/95) to afford a foam (0.090 g). The resulting foam is treated with 2N HCl/Et₂O to afford the title compound as mono salt; mp 153-154° C.; MS (+) ESI: 318 (M+H)⁺; the compound is identified by ¹H NMR. Elemental Analysis for: C₂₀H₁₉N₃O 1.0 HCl 2.1H₂O Calculated: C, 61.33; H, 6.23; N, 10.73 Found: C, 61.01; H, 5.80; N, 10.51

### EXAMPLE 8

### Preparation of Methyl 3-(1H-indol-4-yl)prop-2-enoate

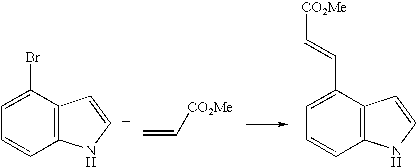

To a solution of 4-bromoindole (1.96 g, 10.0 mmol) in DMF (15 ml) is added methyl acrylate (1.8 ml, 20.0 mmol), palladium(II) acetate (44 mg, 0.2 mmol), triphenyl phospine (104 mg, 0.4 mmol) and N,N-diisopropylethylamine (2.2 ml, 12.5 mmol). After stirring at 100° C. for 3 days, the reaction mixture is cooled to room temperature, treated with aqueous 1 N HCl (50 ml) and extracted with ethyl acetate (3×50 ml). The combined organic extracts are washed with aqueous 1 N HCl (3×50 ml), brine (50 ml), dried (MgSO₄) and concentrated. The crude product is purified by chromatography (silica gel, EtOAc/hexane: 30/70) to afford the title compound as a light yellow solid, (1.66 g, 82%); mp 113-115° C.; MS (−) APCI: 200 (M−H)⁻; the compound is identified by ¹H NMR. Elemental Analysis for: C₁₂H₁₁NO₂ 0.2 H₂O Calculated: C, 70.37; H, 5.61; N, 6.84 Found: C, 70.61; H, 5.46; N, 6.50

### EXAMPLE 9

### Preparation of 3-(1H-Indol4-yl)prop-2-enoic acid

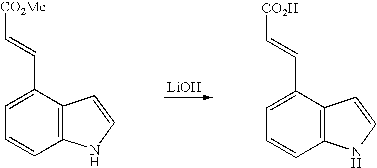

To a solution of methyl 3-(1H-indol-4-yl)prop-2-enoate (5.00 g, 26.6 mmol) in THF (33 ml) is added aqueous 1N LiOH (53 ml). After stirring at room temperature for 18 hours, the reaction mixture is diluted with EtOAc (50 ml). The organic layer is separated and the aqueous layer is neutralized with aqueous 2N HCl and extracted with EtOAc (3×50 ml). The combined organic extracts are washed with brine (50 ml), dried (MgSO₄) and concentrated. The crude product is purified by flash chromatography (silica gel, EtOAc/hexane to EtOAc/MeOH: 50/50 to 95/5) to afford the title compound as a light yellow solid (3.60 g, 72%); mp 217° C. (dec); MS (+) APCI: 188 (M+H)⁺; the compound is identified by ¹H NMR. Elemental Analysis for: C₁₁H₉NO₂ Calculated: C, 70.58; H, 4.85; N, 7.48 Found: C, 70.33; H, 4.69; N, 7.29

### EXAMPLE 10

### Preparation of 3-(1H-Indol-4-yl)prop-2-enoyl azide

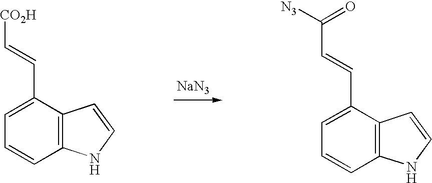

To a solution of triethylamine (3.0 ml, 22.0 mmol) in acetone (18 ml) is added 3-(1 H-indol-4-yl)prop-2-enoic acid (3.48 g, 20.0 mmol). The reaction mixture is maintained below 0° C. while a solution of ethyl chloroformate (2.60 ml, 27.2 mmol) in acetone (18 ml) is added dropwisely. After stirring the mixture at 0° C. for 1 hour, a solution of sodium azide (1.35 g, 30.0 mmol) in H₂O (5 ml) is added in portions. The reaction mixture is stirred at 0° C. for 1 hour. The insoluble material is removed by filtration and the filtrate is concentrated. The residue is dissolved in Et₂O, washed with water, saturated aqueous NaHCO₃, brine, dried over (MgSO₄) and concentrated. The crude product is purified by chromatography (silica gel, EtOAc/hexane: 10/90-30/70) to afford the title compound as a yellow solid (2.7 g, 64%); mp 105-106° C.; MS (−) ESI: 211 (M−H)⁻; the compound is identified by ¹H NMR. Elemental Analysis for: C₁₁H₈N₄O Calculated: C, 62.26; H, 3.80; N, 26.40 Found: C, 62.14; H, 3.79; N, 26.57

### EXAMPLE 11

### Preparation of 3,7-Dihydro-6H-pyrrolo[3,2-f]isoquinolin-6-one

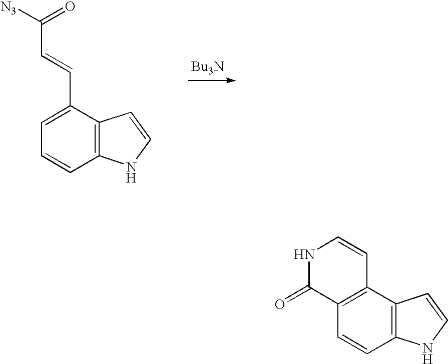

To a solution of Bu₃N (4.1 ml, 17.3 mmol) in Ph₂O (50 ml) at 240° C. is added a solution of 3-(1H-indol-4-yl)prop-2-enoic azide (3.45 g, 16.3 mmol) in Ph₂O (50 ml). After stirring at 210° C. for 30 minutes, the reaction mixture is cooled to room temperature and treated with hexane. The resulting product is collected by filtration and air dried to give the title compound as a yellow solid (2.8 g, 96%); mp >300° C. (dec.); MS (+) ESI: 185 (M+H)⁺; the compound is identified by ¹H NMR. Elemental Analysis for: C₁₁H₈N₂O Calculated: C, 71.73; H, 4.38; N, 15.21 Found: C, 71.39; H, 3.99; N, 14.89

### EXAMPLE 12

### Preparation of 7-(4-Fluorobenzyl)-3,7-dihydro-6H-pyrrolo[3,2-f]isoquinolin-6-one

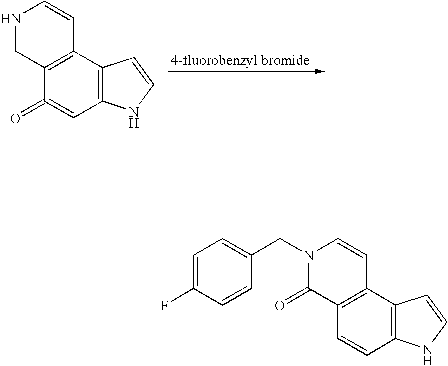

To a solution of 1,7-dihydro-6H-pyrrolo[3,2-f]isoquinolin-6-one (188 mg, 1.02 mmol) in DMF (3 ml) is added K₂CO₃ (276 mg, 2.00 mmol) and 4-fluorobenzyl bromide (0.12 ml, 1.0 mmol). After stirring at room temperature for 18 hours, the reaction mixture is quenched with aqueous 1N HCl. The aqueous is extracted with several portions of EtOAc. The combined organic extracts are washed with aqueous water, brine, dried (MgSO₄) and concentrated. The residue is purified by chromatography (silica gel, EtOAc/hexane: 50/50 to 70/30) to afford the title compound as a yellow solid (199 mg, 68%); mp 171-173° C.; MS (−) ESI, 291 (M−H)⁻; the compound is identified by ¹H NMR. Elemental Analysis for: C₁₈H₁₃FN₂O Calculated: C, 73.96; H, 4.48; N, 9.58 Found: C, 73.52; H, 4.43; N, 9.33

### EXAMPLES 13-15

### Preparation of 3-Aminoalkyl-7-(4-fluorobenzyl)-3.7-dihydro-6H-pyrrolo[3,2-f]isoquinolin-6-one Compounds

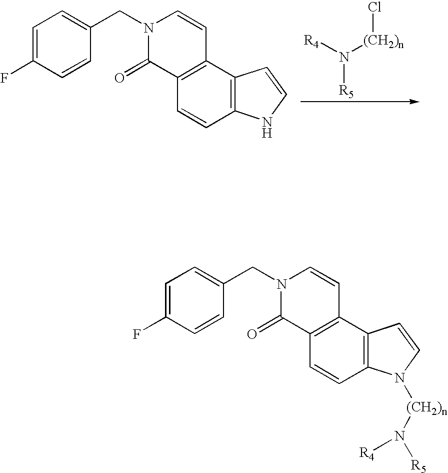

Using essentially the same procedure described for Example and employing the appropriate aminoalkyl chloride and 7-(4-fluorobenzyl)-3,7-dihydro-6H-pyrrolo[3,2-f]isoquinolin-6-one as substrate, the compounds shown in Table I are prepared and identified by mass spectral and NMR analyses.

TABLE I

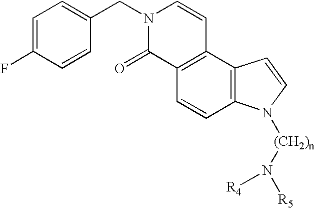

| Ex. No. | n | R4 | R5 | mp° C. | (M + H)⁺ |
|---|---|---|---|---|---|
| 13 | 2 | H | H | 203-205 | 336 |
| 14 | 2 | CH₃ | CH₃ | 221-223 | 364 |
| 15 | 2 | —CH₂—CH₂—CH₂—CH₂— | | 229-231 | 390 |
| 16 | 3 | CH₃ | CH₃ | 49-51 | 378 |

### EXAMPLE 17

### Preparation of tert-Butyl (2S)-2-{[7-(4-fluorobenzyl)-6-oxo-6,7-dihydro-3H-Pyrrolo[3,2-f]isoquinolin-3-yl]methyl}pyrrolidine-1-carboxylate

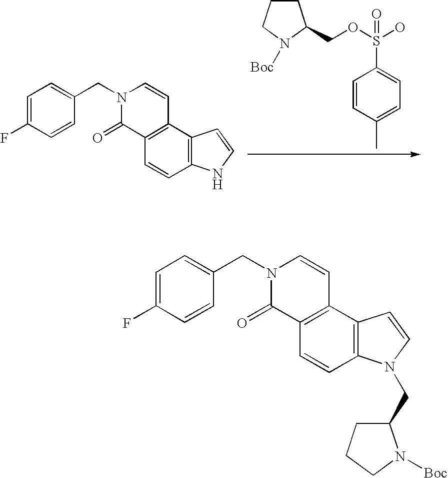

To a solution of 7-(4-fluorobenzyl)-3,7-dihydro-6H-pyrrolo[3,2-f]isoquinolin-6-one (166 mg, 0.57 mmol) in DMF (1.0 ml) is added NaH (27 mg, 0.68 mmol). The reaction mixture was stirred for 30 min at room temperature. To the resulting mixture is added a solution of tert-butyl (2S)-2-({[(4-methylphenyl)sulfonyl]oxy}methyl)pyrrolidine-1-carboxylate (265 mg, 0.75 mmol) in DMF (1.0 ml). After stirring for 18 hours, the reaction mixture is poured to a cooled aqueous 1N HCl. The aqueous is extracted with several portions of EtOAc and the combined organic extracts are washed aqueous 1N HCl, H₂O, brine, dried (MgSO₄) and concentrated on a rotary evaporator. The crude product is purified by chromatography (silica gel, MeOH/CH₂Cl₂: 1/99) to afford the title compound as a off-white solid (270.0 mg, 100%); mp 144-145° C.; MS (+) ESI, 476 (M+H)⁺; [□]_{D}−6.5°(c=3.4, DMSO); the compound is identified by ¹H NMR. Elemental Analysis for: C₂₈H₃₀FN₃O₃ Calculated: C, 70.72; H, 6.36; N, 8.84 Found: C, 70.46; H, 6.34; N, 8.67

### EXAMPLES 19-26

### Preparation of 1-(2-Aminoethyl)-7-substituted-1,7-dihydro-6H-pyrrolo[2,3f]isoquinolin-6-one Compounds

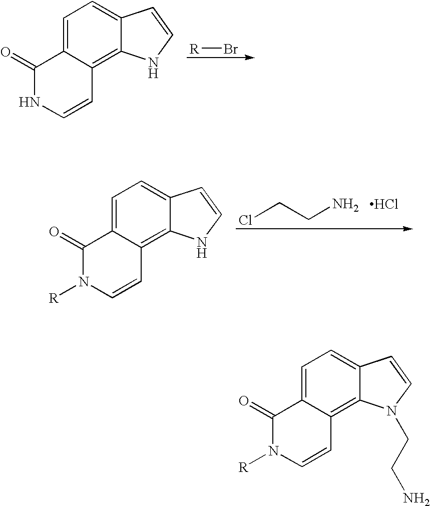

Using essentially the same procedures described in Examples 5 and 7 and employing the desired bromoalkyl reagent, the compounds shown in Table II are obtained and identified by HNMR and mass spectral analyses.

TABLE II

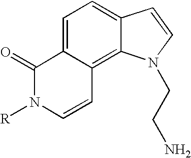

| Ex. No. | R |
|---|---|
| 19 | 1-naphthylmethyl |
| 20 | quinolin-8-ylmethyl |
| 21 | (6-chloroimidazo[2,1-b][1,3]thiazol-5-yl)methyl |
| 22 | (3-chloro-1-benzothien-2-yl)methyl |
| 23 | 2-thienylmethyl |
| 24 | 3-thienylmethyl |
| 25 | 2-chlorobenzyl |
| 26 | 3-chlorobenzyl |

### EXAMPLE 27

### Comparative Evaluation of 5-HT₆ Binding Affinitv of Test Compounds

The affinity of test compounds for the 5-HT₆ receptor is evaluated in the following manner. Cultured Hela cells expressing human cloned 5-HT₆ receptors are harvested and centrifuged at low speed (1,000×g) for 10.0 min to remove the culture media. The harvested cells are suspended in half volume of fresh physiological phosphate buffered saline solution and recentrifuged at the same speed. This operation is repeated. The collected cells are then homogenized in ten volumes of 50 mM Tris.HCl (pH 7.4) and 0.5 mM EDTA. The homogenate is centrifuged at 40,000×g for 30.0 min and the precipitate is collected. The obtained pellet is resuspended in 10 volumes of Tris.HCl buffer and recentrifuged at the same speed. The final pellet is suspended in a small volume of Tris.HCl buffer and the tissue protein content is determined in aliquots of 10-25 microliter volumes. Bovine Serum Albumin is used as the standard in the protein determination according to the method described in Lowry et al., J. Biol. Chem. 1951, 193, 265. The volume of the suspended cell membranes is adjusted to give a tissue protein concentration of 1.0 mg/mL of suspension. The prepared membrane suspension (10 times concentrated) is aliquoted in 1.0 mL volumes and stored at −70° C. until used in subsequent binding experiments.

Binding experiments are performed in a 96 well microtiter plate format, in a total volume of 200 microliters. To each well is added the following mixture: 80.0 microliter of incubation buffer made in 50 mM Tris.HCl buffer (pH 7.4) containing 10.0 mM MgCl₂ and 0.5 mM EDTA and 20 microliters of [³H]-LSD (S.A., 86.0 Ci/mmol, available from Amersham Life Science), 3.0 nM. The dissociation constant, K_{D} of the [³H]-LSD at the human 5-HT₆ receptor is 2.9 nM, as determined by saturation binding with increasing concentrations of [³H]-LSD. The reaction is initiated by the final addition of 100.0 microliters of tissue suspension. Nonspecific binding is measured in the presence of 10.0 micromoles methiothepin. The test compounds are added in 20.0 microliter volume.

The reaction is allowed to proceed in the dark for 120 min at room temperature, at which time, the bound ligand-receptor complex is filtered off on a 96 well unifilter with a Packard Filtermate® 196 Harvester. The bound complex caught on the filter disk is allowed to air dry and the radioactivity is measured in a Packard TopCount® equipped with six photomultiplier detectors, after the addition of 40.0 microliter Microscint®-20 scintillant to each shallow well. The unifilter plate is heat-sealed and counted in a Packard TopCount® with a tritium efficiency of 31%.

Specific binding to the 5-HT₆ receptor is defined as the total radioactivity bound less the amount bound in the presence of 10.0 microliter unlabelled methiothepin. Binding in the presence of varying concentrations of test compound is expressed as a percentage of specific binding in the absence of test compound. The results are plotted as log % bound versus log concentration of the test compound. Nonlinear regression analysis of data points with a computer assisted program Prism® yielded both the IC₅₀ and the Kᵢ values of the test compounds with 95% confidence limits. A linear regression is plotted, from which the IC₅₀ value is determined and the Kᵢ value is determined based upon the following equation:
Kᵢ=IC₅₀/(1+L/K_{D})
where L is the concentration of the radioactive ligand used and K_{D} is the dissociation constant of the ligand for the receptor, both expressed in nM.

Using this assay, the Kᵢ values were determined and are shown in Table III, below.

TABLE III

| Test Compound | 5-HT6 binding Ki |
|---|---|
| (Ex. No.) | (nM) |
| 6 | 388 |
| 7 | 180 |
| 13 | 426 |
| 14 | 379 |
| 15 | 507 |

What is claimed is:

1. A compound of formula I or II 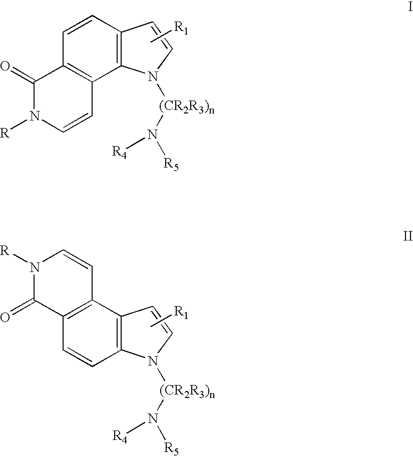
wherein
R is H or a C₁-C₆alkyl, C₁-C₄arylalkyl, C₁-C₄heteroarylalkyl, C₃-C₇cycloalkylalkyl, cycloheteroalkyl, aryl or heteroaryl group each optionally substituted;
R₁ is H, halogen or a C₁-C₆alkoxy or C₁-C₄alkyl group each optionally substituted;
R₂ and R₃ are each independently H or a C₁-C₆alkyl or C₃-C₇cycloalkyl group each optionally substituted or R₂ may be taken together with R₄ to form an optionally substituted 5- to 8-membered ring;
n is 0 or an integer of 1, 2 or 3; and
R₄ and R₅ are each independently H or a C₁-C₆alkyl or C₃-C₇cycloalkyl group each optionally substituted or R₄ and R₅ may be taken together with the atom to which they are attached to form an optionally substituted 5- to 8-membered ring optionally containing an additional heteroatom selected from O, S, or NR; or
a stereoisomer thereof or a pharmaceutically acceptable salt thereof.

2. The compound according to claim 1 wherein R₁ is H.

3. The compound according to claim 1 wherein n is 2 and R₂ and R₃ are H.

4. The compound according to claim 1 wherein R is H, arylalkyl or heteroarylalkyl.

5. The compound according to claim 1 wherein wherein R₄ and R₅ are each independently H or C₁-C₄alkyl.

6. The compound according to claim 2 wherein n is 2 and R₂ and R₃ are H.

7. The compound according to claim 2 wherein R is H, arylalkyl or heteroarylalkyl.

8. The compound according to claim 5 wherein R₁, R₂ and R₃ are H and n is 2.

9. The compound according to claim 1 selected from the group consisting essentially of:
7-benzyl-1-[2-(dimethylamino)ethyl]-1,7-dihydro-6H-pyrrolo[2,3-f]isoquinolin-6-one; 1-(2-aminoethyl)-7-benzyl-1,7-dihydro-6H-pyrrolo[2,3-f]isoquinolin-6-one; 1-(2-aminoethyl)-7-(thien-3-ylmethyl)-1,7-dihydro-6H-pyrrolo[2,3-f]isoquinolin-6-one; 1-(2-aminoethyl)-7-(cyclohexylmethyl)-1,7-dihydro-6H-pyrrolo[2,3-f]isoquinolin-6-one; 3-(2-aminoethyl)-7-(4-fluorobenzyl)-3,7-dihydro-6H-pyrrolo[3,2-f]isoquinolin-6-one; 3-[2-(dimethylamino)ethyl]-7-(4-fluorobenzyl)-3,7-dihydro-6H-pyrrolo[3,2-f]isoquinolin-6-one; 7-(4-fluorobenzyl)-3-(2-pyrrolidin-1-ylethyl)-3,7-dihydro-6H-pyrrolo[3,2-f]isoquinolin-6-one; 3-[3-(dimethylamino)propyl]-7-(4-fluorobenzyl)-3,7-dihydro-6H-pyrrolo[3,2-f]isoquinolin-6-one; or 7-(4-fluorobenzyl)-3-[(2S)-pyrrolidin-2-ylmethyl]-3,7-dihydro-6H-pyrrolo[3,2-f]isoquinolin-6-one; 1-(2-aminoethyl)-7-(1-naphthylmethyl)-1,7-dihydro-6H-pyrrolo[2,3-f]isoquinolin-6-one; 1-(2-aminoethyl)-7-(quinolin-8-ylmethyl)-1,7-dihydro-6H-pyrrolo[2,3-f]isoquinolin-6-one; 1-(2-aminoethyl)-7-[(6-chloroimidazo[2,1-b][1,3]thiazol-5-yl)methyl]-1,7-dihydro-6H-pyrrolo[2,3-f]isoquinolin-6-one; 1-(2-aminoethyl)-7-[(3-chloro-1-benzothien-2-yl)methyl]-1,7-dihydro-6H-pyrrolo[2,3-f]isoquinolin-6-one; 1-(2-aminoethyl)-7-(2-thienylmethyl)-1,7-dihydro-6H-pyrrolo[2,3-f]isoquinolin-6-one; 1-(2-aminoethyl)-7-(2-thienylmethyl)-1,7-dihydro-6H-pyrrolo[2,3-f]isoquinolin-6-one; 1-(2-aminoethyl)-7-(2-chlorobenzyl)-1,7-dihydro-6H-pyrrolo[2,3-f]isoquinolin-6-one; 1-(2-aminoethyl)-7-(3-chlorobenzyl)-1,7-dihydro-6H-pyrrolo[2,3-f]isoquinolin-6-one;
a stereoisomer thereof; and
a pharmaceutically acceptable salt thereof.

10. A method for the treatment of a central nervous system disorder related to or affected by the 5-HT6 receptor in a patient in need thereof which comprises providing to said patient a therapeutically effective amount of a compound of formula I or II 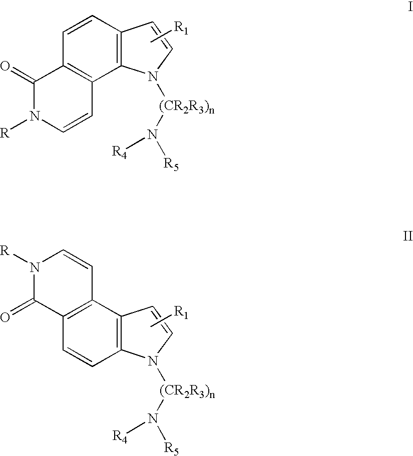
wherein
R is H or a C₁-C₆alkyl, C₁-C₄arylalkyl, C₁-C₄heteroarylalkyl, C₃-C₇cycloalkylalkyl, cycloheteroalkyl, aryl or heteroaryl group each optionally substituted;
R₁ is H, halogen or a C₁-C₆alkoxy or C₁-C₄alkyl group each optionally substituted;
R₂ and R₃ are each independently H or a C₁-C₆alkyl or C₃-C₇cycloalkyl group each optionally substituted or R₂ may be taken together with R₄ to form an optionally substituted 5- to 8-membered ring;
n is 0 or an integer of 1, 2 or 3; and
R₄ and R₅ are each independently H or a C₁-C₆alkyl or C₃-C₇cycloalkyl group each optionally substituted or R₄ and R₅ may be taken together with the atom to which they are attached to form an optionally substituted 5- to 8-membered ring optionally containing an additional heteroatom selected from O, S, or NR; or
a stereoisomer thereof or a pharmaceutically acceptable salt thereof.

11. The method according to claim 10 wherein said disorder is an anxiety disorder or a cognitive disorder.

12. The method according to claim 10 wherein said disorder is a neurodegenerative disorder.

13. The method according to claim 11 wherein said disorder is selected from the group consisting essentially of: attention deficit disorder; obsessive compulsive disorder; withdrawal from drug, alcohol or nicotine addiction; schizophrenia; depression; and Alzheimer's disease.

14. The method according to claim 12 wherein said disorder is selected from the group consisting of: stroke; head trauma; and neuropathic pain.

15. A pharmaceutical composition which comprises a pharmaceutically acceptable carrier and an effective amount of a compound of formula I or II 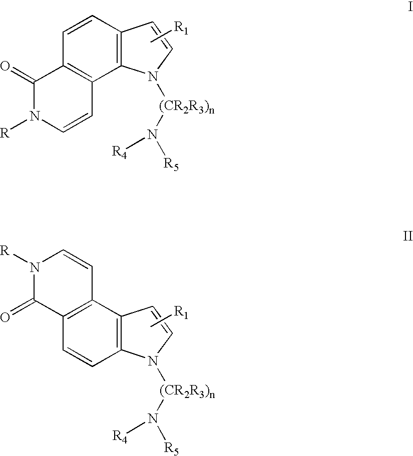
wherein
R is H or a C₁-C₆alkyl, C₁-C₄arylalkyl, C₁-C₄heteroarylalkyl, C₃-C₇cycloalkylalkyl, cycloheteroalkyl, aryl or heteroaryl group each optionally substituted;
R₁ is H, halogen or a C₁-C₆alkoxy or C₁-C₄alkyl group each optionally substituted;
R₂ and R₃ are each independently H or a C₁-C₆alkyl or C₃-C₇cycloalkyl group each optionally substituted or R₂ may be taken together with R₄ to form an optionally substituted 5- to 8-membered ring;
n is 0 or an integer of 1, 2 or 3; and
R₄ and R₅ are each independently H or a C₁-C₆alkyl or C₃-C₇cycloalkyl group each optionally substituted or R₄ and R₅ may be taken together with the atom to which they are attached to form an optionally substituted 5- to 8-membered ring optionally containing an additional heteroatom selected from O, S, or NR; or
a stereoisomer thereof or a pharmaceutically acceptable salt thereof.

16. The composition according to claim 15 having a formula I compound wherein R₁ is H.

17. The composition according to claim 16 having a formula I compound wherein n is 2 and R₂ and R₃ are H

18. The composition according to claim 17 having a formula I compound wherein R is H, arylalkyl or heteroarylalkyl.

19. The composition according to claim 18 having a formula I compound wherein R₄ and R₅ are each independently H or C₁-C₄alkyl.

20. The composition according to claim 15 having a formula I compound selected from the group consisting essentially of:
7-benzyl-1-[2-(dimethylamino)ethyl]-1,7-dihydro-6H-pyrrolo[2,3-f]isoquinolin-6-one; 1-(2-aminoethyl)-7-benzyl-1,7-dihydro-6H-pyrrolo[2,3-f]isoquinolin-6-one; 1-(2-aminoethyl)-7-(thien-3-ylmethyl)-1,7-dihydro-6H-pyrrolo[2,3-f]isoquinolin-6-one; 1-(2-aminoethyl)-7-(cyclohexylmethyl)-1,7-dihydro-6H-pyrrolo[2,3-f]isoquinolin-6-one; 3-(2-aminoethyl)-7-(4-fluorobenzyl)-3,7-dihydro-6H-pyrrolo[3,2-f]isoquinolin-6-one; 3-[2-(dimethylamino)ethyl]-7-(4-fluorobenzyl)-3,7-dihydro-6H-pyrrolo[3,2-f]isoquinolin-6-one; 7-(4-fluorobenzyl)-3-(2-pyrrolidin-1-ylethyl)-3,7-dihydro-6H-pyrrolo[3,2-f]isoquinolin-6-one; 3-[3-(dimethylamino)propyl]-7-(4-fluorobenzyl)-3,7-dihydro-6H-pyrrolo[3,2-f]isoquinolin-6-one; or 1-(2-aminoethyl)-7-(1-naphthylmethyl)-1,7-dihydro-6H-pyrrolo[2,3-f]isoquinolin-6-one; 1-(2-aminoethyl)-7-(quinolin-8-ylmethyl)-1,7-dihydro-6H-pyrrolo[2,3-f]isoquinolin-6-one; 1-(2-aminoethyl)-7-[(6-chloroimidazo[2,1-b][1,3]thiazol-5-yl)methyl]-1,7-dihydro-6H-pyrrolo[2,3-f]isoquinolin-6-one; 1-(2-aminoethyl)-7-[(3-chloro-1-benzothien-2-yl)methyl]-1,7-dihydro-6H-pyrrolo[2,3-f]isoquinolin-6-one; 1-(2-aminoethyl)-7-(2-thienylmethyl)-1,7-dihydro-6H-pyrrolo[2,3-f]isoquinolin-6-one; 1-(2-aminoethyl)-7-(2-thienylmethyl)-1,7-dihydro-6H-pyrrolo[2,3-f]isoquinolin-6-one; 1-(2-aminoethyl)-7-(2-chlorobenzyl)-1,7-dihydro-6H-pyrrolo[2,3-f]isoquinolin-6-one; 1-(2-aminoethyl)-7-(3-chlorobenzyl)-1,7-dihydro-6H-pyrrolo[2,3-f]isoquinolin-6-one;
a stereoisomer thereof; and
a pharmaceutically acceptable salt thereof.
